(19) United States
(12) Patent Application Publication
(10) Pub. No.: US 2006/0089458 A1
(43) Pub. Date: Apr. 27, 2006

(54) Articles suitable for autoclave sterilization

(76) Inventors: John Edward Davis, Westfield, MA (US); Thomas J. Hartle, Delmar, NY (US); Mukund Parthasarathy, Delmar, NY (US); Dennilu Sosa, Pottstown, PA (US)

Correspondence Address: CANTOR COLBURN, LLP, 55 GRIFFIN ROAD SOUTH, BLOOMFIELD, CT, 06002, US

(21) Appl. No.: 10/972,098

(22) Filed: Oct. 22, 2004

(51) Int. Cl. C08L 53/00

(52) U.S. Cl. 525/92 D

(57) ABSTRACT

Articles made from poly(arylene ether) compositions wherein injection molded test specimens having a thickness of 2.5 mm of the compositions exhibit an energy to maximum load of at least 50 kg-cm under ISO 6603 after being subjected to autoclave sterilization with steam heat at 121° C. for 83.2 hours and exhibit an average percent change in energy to maximum load of less than or equal to an absolute value of 10% after 83.2 hours under the same autoclave sterilization conditions. The test specimens are independently prepared using one of injection molding, compression molding, blow molding, sheet extrusion, or thermoformed techniques. Articles include various trays, cages, housings, medical instruments, and other articles that are subjected to steam autoclave sterilization.

### BACKGROUND OF THE INVENTION

This disclosure relates to articles made from poly(arylene ether) compositions, in particular articles useful in medical applications.

In medical and research facilities it is critical that all equipment and materials used for treating patients or housing test specimens are absolutely safe for use to minimize the chances for spreading of diseases. Diseases such as Hepatitis B, are known to be transmitted through contaminated surgical instruments. This has resulted in stricter guidelines for disinfection and sterilization as the weapons to meet the demand for infection control and reduce or eliminate research contamination. Common sterilization methods include the use of dry hot air, autoclaving with moist (steam) heat, gas (ethylene oxide treatment), radiation, and chemical sterilization with a disinfectant such as glutaraldehyde. Although each of these methods has its own specialized uses, autoclaving with moist (steam) heat is the most dependable and widely used procedure for the destruction of microbial life.

Steam sterilization generally denotes heating in an autoclave employing saturated steam under pressure to achieve a chamber temperature of at least 121° C. for periods of 30 minutes or longer. The reliability of this sterilization method is dependent on achieving the proper temperature and time as well as the complete replacement of the air with steam (i.e. no entrapment of air). Stricter sterilization standards combined with the desire to minimize sterilization cycle times has resulted in a trend to increase temperatures of the pressurized steam to 134° C. for time periods under 30 minutes. This type of wet heat is believed to kill most known microbial cells including spores that are normally heat resistant.

There has been an increased use of plastic materials in the overall healthcare and clinical research industries including articles used in the medical, dental, surgical, research and veterinary professions. Plastic materials are often desired as materials of construction over traditional metals, such as stainless steel, for reasons such as material cost reduction, ease of manufacture, design freedom, light weight, dent resistance and aesthetics. Examples of such articles include various instruments and tools such as forceps, probes, directors, retractors, dilators, speculum, scalpels, keratomes, scissors, shears, specula, catheters, hooks, curettes, chisels, clamps, depressors, pliers, extractors, scalers, spatula and the like. Additionally, many articles are made partially from plastic materials, e.g., handles with the remainder formed from traditional materials such as surgical steel. The use of plastic materials has also increased in the manufacture of various trays, containers and sheets that are commonly used to store, house, transfer and cover such instruments and tools as well as other articles used in the healthcare and clinical research industries that require sterilization including instruments trays and containers, waste containers, sample containers, light housings, instrument covers, cages and the like.

The healthcare and clinical research industry trend towards use of increasingly higher temperatures in autoclave sterilization with steam heat has reduced the available selection of suitable plastic materials. The articles, including trays, covers, cages and containers, need to be able to withstand repeated exposure to the sterilization process without embrittlement or significant loss of physical properties. Articles that become brittle and shatter into numerous pieces are generally unacceptable, especially in surgical rooms where all articles need to be accounted for after a surgical procedure is completed. The combination of desired physical property attributes and the increasingly demanding sterilization techniques have resulted in an ongoing need for articles made from plastic materials that exhibit enhanced physical properties.

### BRIEF DESCRIPTION OF THE INVENTION

The needs for improved articles described above are met, at least in part, by articles made from poly(arylene ether) compositions wherein test specimens having a thickness of 2.5 millimeters (mm) of the compositions exhibit an energy to maximum load greater than or equal to kilogram-centimeters (70 kg-cm) under ISO 6603 after being subjected to autoclave sterilization with steam heat at 134° C. for 20 hours and exhibit an average percent change in energy to maximum load of less than or equal to an absolute value of 10% after 80 hours under the same autoclave sterilization conditions. The test specimens may be prepared using one of injection molding, compression molding, blow molding, sheet extrusion, profile extrusion or thermoforming techniques.

### DETAILED DESCRIPTION

In another embodiment, articles are made from compositions comprising at least one poly(arylene ether), at least one nonelastomeric polymer of an alkenylaromatic compound, and at least one impact modifier; wherein test specimens having a thickness of 2.5 mm of the compositions exhibit an energy to maximum load greater than or equal to 70 kg-cm under ISO 6603 after being subjected to autoclave sterilization with steam heat at 134° C. for 20 hours and exhibit an average percent change in energy to maximum load of less than or equal to an absolute value of 10% after 80 hours under the same autoclave sterilization conditions. The test specimens may be prepared using one of injection molding, compression molding, blow molding, sheet extrusion, profile extrusion or thermoforming techniques.

In another embodiment, articles are made from compositions comprising at least one poly(arylene ether), at least one rubber-modified polystyrene, and at least one hydrogenated block copolymer; wherein test specimens having a thickness of 2.5 mm of the compositions exhibit an energy to maximum load greater than or equal to 70 kg-cm under ISO 6603 after being subjected to autoclave sterilization with steam heat at 134° C. for 20 hours and exhibit an average percent change in energy to maximum load of less than or equal to an absolute value of 10% after 80 hours under the same autoclave sterilization conditions. The test specimens may be prepared using one of injection molding, compression molding, blow molding, sheet extrusion, profile extrusion or thermoforming techniques.

It is contemplated that in the ever changing anti-microbial environment future changes to sterilization techniques may include temperatures greater than 134° C. and optionally time less than 30 minutes. It is anticipated that the articles and materials described herein would be useful in sterilization techniques employing temperatures greater than 134° C. but less than or equal to 175° C.

The poly(arylene ether)s utilized in resin compositions are known polymers having structural units of formula I. 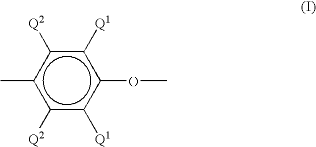
wherein each Q¹ is independently halogen, primary or secondary lower alkyl, phenyl, haloalkyl, aminoalkyl, hydrocarbonoxy, or halohydrocarbonoxy wherein at least two carbon atoms separate the halogen and oxygen atoms; each Q² is independently hydrogen, halogen, primary or secondary lower alkyl, phenyl, haloalkyl, hydrocarbonoxy or halohydrocarbonoxy as defined for Q¹.

Both homopolymer and copolymer poly(arylene ether)s are included. The preferred homopolymers include those containing 2,6-dimethyl-1,4-phenylene ether units. Suitable copolymers include copolymers containing such 2,6-dimethyl-1,4-phenylene ether units in combination with, for example, 2,3,6-trimethyl-1,4-phenylene ether units. Also included are poly(arylene ether)s containing moieties prepared by grafting onto the poly(arylene ether) in known manners such materials as vinyl monomers or polymers such as polystyrenes and elastomers, as well as coupled poly(arylene ether)s in which coupling agents such as low molecular weight polycarbonates, quinones, heterocycles and formals undergo reaction in known manner with the hydroxy groups of two poly(arylene ether) chains to produce a higher molecular weight polymer, provided a substantial proportion of free OH groups remains.

The poly(arylene ether)s generally have an intrinsic viscosity greater than or equal to 0.25, often 0.25 to 0.6, and more specifically 0.35 to 0.60 deciliters per gram (dl./g.), as measured in chloroform at 25° C. It is also possible to utilize a high intrinsic viscosity poly(arylene ether) and a low intrinsic viscosity poly(arylene ether) in combination. Such low intrinsic viscosity poly(arylene ether) may have an intrinsic viscosity of 0.10 to 0.33 dl/g as measured in chloroform at 25° C. Determining an exact ratio, when two intrinsic viscosities are used, will depend somewhat on the exact intrinsic viscosities of the poly(arylene ether) used and the ultimate physical properties that are desired. The poly(arylene ether) can have a number average molecular weight of about 3,000 to about 40,000 grams per mole (g/mol) and/or a weight average molecular weight of about 5,000 to about 80,000 g/mol, as determined by gel permeation chromatography using monodisperse polystyrene standards, a styrene divinyl benzene gel at 40° C. and samples having a concentration of 1 milligram per milliliter of chloroform.

Poly(arylene ether)s are typically prepared by the oxidative coupling of at least one monohydroxyaromatic compound such as 2,6-xylenol or 2,3,6-trimethylphenol. Catalyst systems are generally employed for such coupling; they typically contain at least one heavy metal compound such as a copper, manganese or cobalt compound, usually in combination with various other materials.

The poly(arylene ether)s include those which comprise molecules having at least one aminoalkyl-containing end group. The aminoalkyl radical is covalently bound to a carbon atom located in an ortho position to the hydroxy group. Products containing such end groups may be obtained by incorporating an appropriate primary or secondary monoamine such as di-n-butylamine or dimethylamine as one of the constituents of the oxidative coupling reaction mixture. Also frequently present are 4-hydroxyalkylsubstituted biphenyl end groups and/or alkylsubstituted biphenyl structural units, typically obtained from reaction mixtures in which a by-product alkylsubstituted diphenoquinone, e.g., tetramethylhydroquinone from 2,6-xylenol, is present, especially in a copper-halide-secondary or tertiary amine system. A substantial proportion of the polymer molecules, typically constituting as much as about 90% by weight of the polymer, may contain at least one of said aminoalkyl-containing and alkylsubstituted 4-hydroxybiphenyl end groups.

In one embodiment, the poly(arylene ether) comprises a capped poly(arylene ether). The capping may be used to reduce the oxidation of terminal hydroxy groups on the poly(arylene ether) chain. The terminal hydroxy groups may be inactivated by capping with an inactivating capping agent via an acylation reaction, for example. The capping agent chosen is desirably one that results in a less reactive poly(arylene ether) thereby reducing or preventing crosslinking of the polymer chains and the formation of gels or black specks during processing at elevated temperatures. Suitable capping agents include, for example, esters of salicylic acid, anthranilic acid, or a substituted derivative thereof, and the like; esters of salicylic acid, and especially salicylic carbonate and linear polysalicylates, are preferred. As used herein, the term “ester of salicylic acid” includes compounds in which the carboxy group, the hydroxy group, or both have been esterified. Suitable salicylates include, for example, aryl salicylates such as phenyl salicylate, acetylsalicylic acid, salicylic carbonate, and polysalicylates, including both linear polysalicylates and cyclic compounds such as disalicylide and trisalicylide. The preferred capping agents are salicylic carbonate and the polysalicylates, especially linear polysalicylates. When capped, the poly(arylene ether) may be capped to any desirable extent up to 80 percent, more specifically up to about 90 percent, and even more specifically up to 100 percent of the hydroxy groups are capped. Suitable capped poly(arylene ether) and their preparation are described in U.S. Pat. No. 4,760,118 to White et al. and U.S. Pat. No. 6,306,978 to Braat et al.

Capping poly(arylene ether) with polysalicylate is also believed to reduce the amount of aminoalkyl terminated groups present in the poly(arylene ether) chain. The aminoalkyl groups are the result of oxidative coupling reactions that employ amines in the process to produce the poly(arylene ether). The aminoalkyl group, ortho to the terminal hydroxy group of the poly(arylene ether), is susceptible to decomposition at high temperatures. The decomposition is believed to result in the regeneration of primary or secondary amine and the production of a quinone methide end group, which may in turn generate a 2,6-dialkyl-1-hydroxyphenyl end group. Capping of poly(arylene ether) containing aminoalkyl groups with polysalicylate is believed to remove such amino groups to result in a capped terminal hydroxy group of the polymer chain and the formation of 2-hydroxy-N,N-alkylbenzamine (salicylamide). The removal of the amino group and the capping provides a poly(arylene ether) that is more stable to high temperatures, thereby resulting in fewer degradative products, such as gels or black specks, during processing of the poly(arylene ether).

Also useful in some embodiments are poly(arylene ether)s which have been functionalized by the reaction of the poly(arylene ether) with a functionalizing agent such as maleic anhydride, citric acid, fumaric acid, a derivative of the foregoing, functional equivalent of the foregoing, or a combination comprising two or more of the foregoing.

It will be apparent to those skilled in the art from the foregoing that the poly(arylene ether)s include many of those presently known, irrespective of variations in structural units or ancillary chemical features.

In some embodiments, particularly those in which the article comprising the composition will be in contact with food, the poly(arylene ether) may have residual levels of reagents and side products from the manufacture of the poly(arylene ether) below certain levels. Accordingly, the poly(arylene ether) may described by one or more of the following limits expressed as weight percent with respect to the weight of the poly(arylene ether): (a) less than or equal to 0.16 weight percent diethylamine, (b) less than or equal to 0.02 weight percent methyl alcohol, and (c) less than or equal to 0.2 weight percent toluene. The amounts of diethylamine, methyl alcohol and toluene may be determined using gas chromatography, optionally in combination with mass spectrometry.

The resin composition may comprise poly(arylene ether) in amounts that vary over a wide range. Most often the poly(arylene ether) is employed in an amount sufficient to avoid distortion of the article from the high temperatures utilized during sterilization, generally in an amount greater than or equal to 40%, in some embodiments in an amount greater than or equal to 50%, and in some other embodiments in an amount greater than or equal to 60% by weight, and in some additional embodiments in an amount greater than or equal to 65% by weight based on the total weight of the composition. The upper limit of the amount of the poly(arylene ether) is generally less than or equal to 95%, in some embodiments less than or equal to 90%, and in some other embodiments less than or equal to 80% by weight, based on the total weight of the composition. In one embodiment the amount of poly(arylene ether) is sufficient to afford a composition having a deflection temperature under load determined according to ISO 75 greater than or equal to 130° C. under a load of 1.80 MPa measured flatwise. In another embodiment the amount of poly(arylene ether) is sufficient to afford a composition having a deflection temperature under load determined according to ISO 75 greater than or equal to 140° C. under a load of 1.80 MPa measured flatwise.

Nonelastomeric polymers of an alkenylaromatic compound may be prepared by methods known in the art including bulk, suspension and emulsion polymerization. They generally contain at least about 40% by weight of structural units derived from an alkenylaromatic monomer of the formula (II): 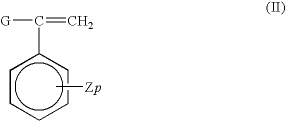
wherein G is hydrogen, lower alkyl or halogen; Z is vinyl, halogen or lower alkyl; and p is from 0 to 5. These resins include homopolymers of styrene, chlorostyrene and vinyltoluene, random copolymers of styrene with one or more monomers illustrated by acrylonitrile, butadiene, α-methylstyrene, ethylvinylbenzene, divinylbenzene and maleic anhydride, and rubber-modified polystyrenes comprising blends and grafts, wherein the rubber is a polybutadiene or a rubbery copolymer of 98-68% styrene and 2-32% diene monomer. These rubber modified polystyrenes include high impact polystyrene (commonly referred to as HIPS). In one embodiment, the high impact polystyrene contains an ethylene-propylene rubber or an ethylene-propylene-diene rubber. Non-elastomeric block copolymer compositions of styrene and butadiene can also be used that have linear block, radial block or tapered block copolymer architectures. They are commercially available from such companies as Total Petrochemicals as under the trademark FINACLEAR and Phillips under the trademark K-RESINS.

In some embodiments, the nonelastomeric alkenylaromatic polymer compound used in the resin compositions has a less than or equal to 0.5 weight percent of total residual styrene monomer with respect to the total weight of the nonelastomeric alkenylaromatic polymer, more specifically a less than or equal to 0.2 weight percent of total residual styrene monomer. The total residual styrene monomer content can be determined by methods well known in the art, such as those described in the United States Code of Federal Regulations, Title 21, Volume 3, Section 177.1640 (revised as of Apr. 1, 2004).

The amount of nonelastomeric alkenylaromatic polymer compound used in the resin compositions is an amount effective to improve the flow and processability of the composition compared to an analogous composition without the nonelastomeric alkenylaromatic polymer. Improved flow can be indicated by reduced viscosity or reduced injection pressures needed to fill a tool to manufacture an article during an injection molding process. The upper limit in the amount of the nonelastomeric alkenylaromatic polymer compound that may be utilized is largely dictated by the maximum temperature that the article will be exposed to during sterilization process so as to avoid distortion during sterilization as well as the article design and physical property requirements, including the desired melt viscosity for manufacture of the article. Generally, the nonelastomeric alkenylaromatic polymer is utilized in an amount of less than or equal to 60%, in some embodiments less than or equal to 50%, and in some other embodiments in an amount less than or equal to 40% by weight based on the total weight of the composition. The lower limit of the amount of the nonelastomeric alkenylaromatic polymer is generally greater than or equal to 1%, in some embodiments, greater than or equal to 10%, and in some other embodiments greater than or equal to 20% by weight based on the total weight of the composition.

The resin compositions also contain at least one impact modifier. The impact modifiers include block (typically multi-block, e.g., diblock, triblock and greater, or radial teleblock) copolymers of alkenyl aromatic compounds and dienes. Most often at least one block is derived from styrene and at least one block from at least one of butadiene and isoprene. Especially useful are the multi-block, e.g., triblock and diblock, copolymers comprising polystyrene blocks and diene derived blocks. Useful impact modifiers include those that contain less than or equal to 70% by weight, in some embodiments less than or equal to 50% by weight, and more specifically less than or equal to 40% by weight, of an alkenyl aromatic compound, typically styrene, with the remainder derived from dienes. In one embodiment, the aliphatic unsaturation residue from the dienes has been preferentially removed with hydrogenation. The weight average molecular weights of the impact modifiers are typically 50,000 to 300,000. Block copolymers of this type are generally referred to as SBS, S-EB-S, and S-EP copolymers and are available commercially from a number of sources, including Phillips Petroleum under the trademark SOLPRENE, Kraton Polymers under the trademark KRATON, Kuraray Company, Ltd. under the trademark SEPTON and Asahi Kasei Chemical Corp. under the trademark TUFTEC.

Various mixtures of the aforementioned impact modifiers may also be useful. The amount of the impact modifier generally present is an amount effective to improve the physical properties, for example, the ductility of an article made from the composition when compared to an article made from the same composition without an impact modifier. Improved ductility can be indicated by increased impact strength, increased tensile elongation to break, or both increased impact strength and increased tensile elongation to break. Generally, the amount of impact modifier is 1% to 20% by weight based on the total weight of the composition. In some embodiments the range is 1% to 10% by weight; based on the total weight of the composition. The exact amount and types or combinations of impact modifiers utilized will depend in part on the requirements needed in the article molded from the resin composition.

The resin compositions may also comprise additives. Possible additives include anti-oxidants, drip retardants, flame retardants, dyes, pigments, colorants, stabilizers, small particle minerals (e.g., clay, mica, talc, and the like), glass fibers including long glass fibers, glass beads, antistatic agents, plasticizers, lubricants, and combinations comprising at least one of the foregoing additives. These additives are known in the art, as are their effective levels and methods of incorporation. Exemplary additives include hindered phenols, zinc oxide, and zinc sulfide. Effective amounts of the additives vary widely, but they are usually present in an amount less than or equal to 20 weight percent (wt %) or so, based on the total weight of the composition. In some embodiments the amount of these additives, individually, is about 0.1 wt % to about 5 wt %, based upon the total weight of the composition although other ranges may be preferred for some articles.

The resin compositions can be prepared by a variety of methods involving intimate admixing of the materials with any additional additives desired in the formulation. Suitable procedures include solution blending and melt blending. Because of the availability of melt blending equipment in commercial polymer processing facilities, melt processing procedures are generally preferred. Examples of equipment used in such melt compounding methods include: co-rotating and counter-rotating extruders, single screw extruders, disc-pack processors and various other types of extrusion equipment. In some instances, the compounded material exits the extruder through small exit holes in a die and the resulting strands of molten resin are cooled by passing the strands through a water bath. The cooled strands can be chopped into small pellets for packaging and further handling.

All of the ingredients may be added initially to the processing system, or else certain additives may be pre-compounded with each other. It is sometimes advantageous to introduce liquid components, e.g., flow promoters and flame retardants, into the compounder through the use of a liquid injection system as is known in the compounding art. It is generally advantageous to employ at least one vent port (either atmospheric or vacuum) to allow venting of the melt. Those of ordinary skill in the art will be able to adjust blending times and temperatures, as well as component addition location and sequence, without undue additional experimentation.

The resin compositions are converted to the articles using common thermoplastic processes such as injection molding, gas-assist injection molding, film and sheet extrusion, profile extrusion, pultrusion, extrusion molding, compression molding and blow molding. Film and sheet extrusion processes may include and are not limited to melt casting, blown film extrusion and calendaring. Co-extrusion and lamination processes may be employed to form composite multi-layer films or sheets.

The films and sheets described above may further be thermoplastically processed into shaped articles via forming and molding processes including but not limited to thermoforming, vacuum forming, pressure forming, injection molding and compression molding. Multi-layered shaped articles may also be formed by injection molding a thermoplastic resin onto a single or multi-layer film or sheet substrate.

The articles include sterilization trays for surgical, orthopedic, dental, and/or veterinary instruments, animal cages as well as general pharma, clinical research and lab trays and include independently lids, bottoms and inner insert compartments of such trays. The size of these trays can vary over a wide range, generally from 2.5×5×5 cm to 60×45×15 cm, although other tray sizes are also contemplated. Other articles include veterinary trays and cages which can vary in size from very small, e.g., 10×15×25 cm to very large, e.g., 120×120×120 cm. Various food trays, food containers and food packaging that require autoclave sterilization as described herein are included. These articles-can also vary in size, e.g., from 2.5×5×15 cm to 10×30×45 cm. Membrane filtration media, including piping and tubing, and the associated housings that require sterilization are within the scope of the envisioned articles. Various medical instrument components and handles, e.g., internal components, levers, triggers, handles, housings, and the like, are also contemplated.

Those skilled in the art will also appreciate that common curing and surface modification processes including and not limited to heat-setting, texturing, embossing, corona treatment, flame treatment, plasma treatment and vacuum deposition may further be applied to the above articles to alter surface appearances and impart additional functionalities to the articles.

The following non-limiting examples are provided to illustrate some embodiments. All percentages are by weight based on the total weight of the entire composition, unless otherwise indicated.

### EXAMPLES

The materials used to prepare the test specimens of the illustrative examples are described in Table I.

TABLE 1

| Material | |
|---|---|
| Name | Material Description/Supplier |
| PPE-1 | A poly(2,6-dimethylphenylene ether) with an intrinsic viscosity of 0.46 dl/g as |
| | measured in chloroform at 25° C. and has a glass transition temperature of 205- |
| | 210° C.; commercially available from GE Advanced Materials. |
| PPE-2 | A poly(2,6-dimethylphenylene ether) with an intrinsic viscosity of 0.40 dl/g as |
| | measured in chloroform at 25° C. and has a glass transition temperature of 205- |
| | 210° C.; commercially available from GE Advanced Materials. |
| PPE-3 | A poly(2,6-dimethylphenylene ether) with an intrinsic viscosity of 0.33 dl/g as |
| | measured in chloroform at 25° C. and has a glass transition temperature of 182- |
| | 188° C.; commercially available from GE Advanced Materials. |
| PPE-4 | A poly(2,6-dimethylphenylene ether) with an intrinsic viscosity of 0.12 dl/g as |
| | measured in chloroform at 25° C. and has a glass transition temperature of 158- |
| | 162° C.; commercially available from GE Advanced Materials. |
| SEBS | Polystyrene-poly(ethylene-butylene)-polystyrene impact modifier commercially |
| | available from Kraton Polymers as G1651. |
| HIPS | A nonelastomeric alkenylaromatic polymer of polybutadiene modified |
| | polystyrene having a polybutadiene content of about 10%; available from GE |
| | Advanced Materials. |
| XPS-1 | A polystyrene homopolymer obtained from Nova as grade 1210. |
| XPS-2 | A polystyrene homopolymer obtained from Huntsman as grade PP738. |
| PS | A polystyrene-polybutadiene copolymer having a styrene content of about 75% |
| | by weight commercially available from Total Petrochemicals as Finaclear 520. |
| PSU | 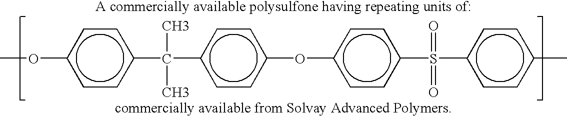 |
| PPSU | 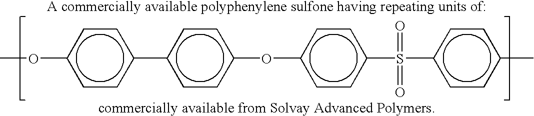 |
| PEI | 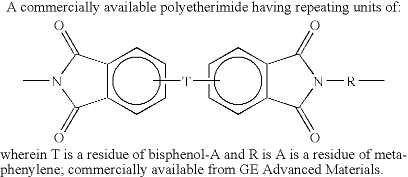 |

Poly(arylene ether) resin containing compositions were prepared according to the compositions indicated in Table 2 by admixing the ingredients in a twin-screw extruder operated at 290° C. and the extrudate was chopped into pellets and dried. The compositions also contain less than 1 weight percent stabilizers and anti-oxidants. Weight percent, as used in the compositions, was determined based on the total weight of the composition.

TABLE 2

| | Example 1 | Example 2 | Example 3 | Example 4 |
|---|---|---|---|---|
| PPE-1 | 70 | — | 70 | — |
| PPE-2 | — | 70 | — | — |
| PPE-3 | — | — | — | 60 |
| PPE-4 | — | — | — | 20 |
| HIPS | 25 | — | — | 20 |
| XPS-1 | — | 10 | — | — |
| XPS-2 | — | — | 30 | — |
| PS | — | 20 | — | — |
| SEBS | 5 | — | — | — |

Various injection molded test specimens having a thickness of 2.5 mm were prepared and subjected to autoclave sterilization with steam heat for the indicated number of hours at 121° C. as outlined in Table 3. Test specimens may alternatively be prepared by one of compression molding, blow molding, sheet extrusion, or thermoformed techniques. These techniques are well-known in the art as are the useful conditions for preparing the test specimens. The test specimens were impacted according to ISO 6603 and the energy to maximum load in kg-cm is presented. A minimum of seven test specimens was impacted after each time period to arrive at an average energy to maximum load value.

TABLE 3

| | 0 hours | 83.3 | 166.7 | 250 | 333.3 |
|---|---|---|---|---|---|
| Test specimen | (control) | hours | hours | hours | hours |
| PSU | 117.3 | 109.1 | 116.0 | 115.0 | 108.6 |
| PPSU | 145.6 | 126.7 | 120.4 | 121.2 | 119.1 |
| PEI | 164.6 | 42.3 | 49.6 | 11.3 | 10.1 |
| Example 1 | 97.4 | 97.6 | 90.1 | 96.1 | 96.1 |
| Example 2 | 93.0 | 49.0 | 40.4 | 34.9 | 40.9 |
| Example 3 | 9.2 | 6.5 | 6.1 | 7.1 | 7.9 |
| Example 4 | 72.3 | 17.4 | 18.3 | 18.5 | 11.7 |

The average percent change in the energy to maximum load for the data presented in Table 3 as compared to the control (before subjecting the test specimens to autoclave sterilization conditions at 121° C.) are presented in Table 4. The average percent change was calculated as the difference between the energy to maximum load after the specified time period and the control followed by dividing the difference by the energy to maximum load of the control. The resulting value was multiplied by one hundred to arrive at the reported values.

TABLE 4

| Test specimen | 83.3 hours | 166.7 hours | 250 hours | 333.3 hours |
|---|---|---|---|---|
| PSU | −7.0 | −1.1 | −2.0 | −7.4 |
| PPSU | −13.0 | −17.3 | −16.8 | −18.2 |
| PEI | −74.3 | −69.9 | −93.1 | −93.9 |
| Example 1 | 0.2 | −7.5 | −1.3 | −1.3 |
| Example 2 | −47.3 | −56.6 | −62.5 | −56.0 |
| Example 3 | −29.3 | −33.7 | −22.8 | −14.1 |
| Example 4 | −75.9 | −74.7 | −74.4 | −83.8 |

As observed by the data in Tables 3 and 4, the high performance materials, PSU, PPSU, and PEI, that are generally used in the manufacture of articles that are subjected to extreme sterilization conditions exhibit high (>50 kg-cm) maximum load initially with an undesirable deterioration after being subjected to the sterilization conditions. This diminishment of properties results in limited useful cycle times for articles made from these materials due to the risks of breakage and brittle failures. The PSU and PPSU materials are generally accepted as the highest performance materials for use in articles that are subjected to autoclave sterilization and represent acceptable performance targets for alternative materials. Example 1 unexpectedly exhibits the desirable combination of a very high (>50 kg-cm) energy to maximum load initially and after extended exposure to autoclave sterilization conditions as indicated by the very low average percent change after 333.3 hours of exposure (1000 equivalent sterilization cycles of 20 minutes). Example 2, Example 3, and Example 4 exhibit unacceptably low energy to maximum load or have the expected too high a level of deterioration of properties after the demanding autoclave sterilization conditions. Comparison of these data highlights the unexpected nature of the results obtained for Example 1, especially when compared to the standard of PSU.

Various injection molded test specimens having a thickness of 2.5 mm were prepared and subjected to autoclave sterilization with steam heat for the indicated number of hours at 134° C. as outlined in Table 5. A minimum of seven test specimens was impacted after each time period to arrive at an average energy to maximum load value. The test specimens were impacted according to ISO 6603 and the energy to maximum load in kg-cm is presented.

TABLE 5

| | 0 hours | 20.8 | 41.6 | 62.4 | 83.2 |
|---|---|---|---|---|---|
| Test specimen | (control) | hours | hours | hours | hours |
| PSU | 117.3 | 109.9 | 99.9 | 100.8 | 76.2 |
| PES | 133.3 | 97.9 | 126.4 | 106.1 | 116.9 |
| PPSU | 145.6 | 129.0 | 132.1 | 131.9 | 126.7 |
| PEI | 161.5 | 37.3 | 8.3 | 11.6 | 12.6 |
| Example 1 | 97.4 | 78.8 | 100.4 | 81.5 | 80.2 |

The average percent change in the energy to maximum load for the data presented in Table 5 as compared to the control (before subjection to autoclave sterilization conditions at 134° C.) are presented in Table 6.

TABLE 6

| Test specimen | 20.8 hours | 41.6 hours | 62.4 hours | 83.2 hours |
|---|---|---|---|---|
| PSU | −6.3 | −14.9 | −14.0 | −35.0 |
| PES | −26.6 | −5.2 | −20.4 | −12.3 |
| PPSU | −11.4 | −9.3 | −9.4 | −13.0 |
| PEI | −76.9 | −94.9 | −92.8 | −92.2 |
| Example 1 | −19.0 | 3.1 | −16.3 | −17.6 |

Described herein are articles made from poly(arylene ether) compositions wherein test specimens of the compositions exhibit an energy to maximum load of at least 50 kg-cm under ISO 6603 after being subjected to autoclave sterilization with steam heat at 134° C. for 20.8 hours and exhibit an average percent change in energy to maximum load of less than 30% after 20.8 hours under the same autoclave sterilization conditions (1000 equivalent sterilization cycles of 5 minutes). In another embodiment, the aforementioned energy to maximum load is at least 70 kg-cm after being subjected to autoclave sterilization with steam heat at 134° C. for 20.8 hours. In another embodiment, the aforementioned average percent change in energy to maximum load is less than 20% after 83.2 hours under the same autoclave sterilization conditions.

While the invention has been described with reference to various embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. An article comprising a resin composition,
wherein the resin composition comprises (a) at least one poly(arylene ether), (b) at least one nonelastomeric polymer of an alkenylaromatic compound, and (c) at least one impact modifier;
wherein a test specimen of 2.5 mm thickness of the resin composition has an energy to maximum load of at least 50 kg-cm measured according to ISO 6603 after exposure to autoclave sterilization with steam heat at 121° C. for 83.3 hours, and wherein a test specimen of 2.5 mm thickness of the resin composition has a percent change in energy to maximum load of less than or equal to an absolute value of 10% when the energy to maximum load is measured according to ISO 6603 before and after exposure to autoclave sterilization with steam heat at 121° C. for 83.3 hours, and
wherein the test specimens are prepared using one of injection molding, compression molding, blow molding, sheet extrusion, or thermoformed techniques.

2. The article of claim 1, wherein the test specimen has at least one of the following:
(i) an energy to maximum load greater than or equal to 70 kg-cm measured according to ISO 6603 after exposure to autoclave sterilization with steam heat at 121° C. for 83.3 hours;
(ii) a percent change in energy to maximum load of less than or equal to an absolute value of 5% when the energy to maximum load is measured according to ISO 6603 before and after exposure to autoclave sterilization with steam heat at 121° C. for 83.3 hours;
(iii) an energy to maximum load greater than or equal to 50 kg-cm measured according to ISO 6603 after exposure to autoclave sterilization with steam heat at 121° C. for 333.3 hours; and
(iv) a percent change in energy to maximum load of less than or equal to an absolute value of 10% when the energy to maximum load is measured according to ISO 6603 before and after exposure to autoclave sterilization with steam heat at 121° C. for 333.3 hours.

3. The article of claim 1, wherein the poly(arylene ether) comprises 2,6-dimethyl-1,4-phenylene ether units, and wherein the poly(arylene ether) comprises a poly(arylene ether) having an intrinsic viscosity of 0.25 to 0.6 dl/g as measured in chloroform at 25° C.

4. The article of claim 1, wherein the nonelastomeric polymer of an alkenylaromatic compound comprises at least one of polystyrene, impact modified polystyrene, or a non-elastomeric block copolymer composition of styrene and butadiene.

5. The article of claim 1, wherein the impact modifier is a block copolymer of an alkenyl aromatic compound and a diene; wherein the alkenyl aromatic compound is less that 70% by weight of the impact modifier.

6. The article of claim 1, wherein the composition comprises:
(a) 40 to 95 weight percent at least one poly(arylene ether),
(b) 1 to 60 weight percent of at least one nonelastomeric polymer of an alkenylaromatic compound, and
(c) 1 to 20 weight percent of at least one impact modifier wherein the weight percents are based on the total weight of the composition.

7. The article of claim 1, wherein the composition comprises:
(a) 65 to 95 weight percent at least one poly(arylene ether),
(b) 1 to 35 weight percent of at least one nonelastomeric polymer of an alkenylaromatic compound, and
(c) 1 to 20 weight percent of at least one impact modifier wherein the weight percents are based on the total weight of the composition.

8. The article of claim 1, wherein the composition has a deflection temperature under load determined according to ISO 75 of at least 130° C. under a load of 1.80 MPa measured flatwise.

9. The article of claim 1, wherein the article is a tray useful for sterilization of surgical, orthopedic, dental, and/or veterinary instruments.

10. The article of claim 1, wherein the article is one of a medical instrument component or a medical instrument handle.

11. An article comprising a resin composition,
wherein the resin composition comprises (a) at least one poly(arylene ether), (b) at least one nonelastomeric polymer of an alkenylaromatic compound, and (c) at least one impact modifier;
wherein an injection molded test specimen of 2.5 mm thickness of the resin composition has an energy to maximum load of at least 50 kg-cm measured according to ISO 6603 after exposure to autoclave sterilization with steam heat at 134° C. for 20.8 hours, and
wherein an injection molded test specimen of 2.5 mm thickness of the resin composition has a percent change in energy to maximum load of less than or equal to an absolute value of 30% when the energy to maximum load is measured according to ISO 6603 before and after exposure to autoclave sterilization with steam heat at 134° C. for 20.8 hours;
wherein the test specimens are prepared using one of injection molding, compression molding, blow molding, sheet extrusion, or thermoformed techniques.

12. The article of claim 11, wherein the test specimen of the resin composition has at least one of the following:
(i) an energy to maximum load of less than 70 kg-cm when measured according to ISO 6603 after exposure to autoclave sterilization with steam heat at 134° C. for 20.8 hours,
(ii) a percent change in energy to maximum load of less than or equal to an absolute value of 20% when the energy to maximum load is measured according to ISO 6603 before and after exposure to autoclave sterilization with steam heat at 134° C. for 20.8 hours,
(iii) an energy to maximum load of at least 70 kg-cm measured according to ISO 6603 after exposure to autoclave sterilization with steam heat at 134° C. for 83.2 hours, and
(iv) a percent change in energy to maximum load of less than or equal to an absolute value of 20% when the energy to maximum load is measured according to ISO 6603 before and after exposure to autoclave sterilization with steam heat at 134° C. for 83.2 hours.

13. The article of claim 11, wherein the poly(arylene ether) comprises 2,6-dimethyl-1,4-phenylene ether units, and wherein the poly(arylene ether) comprises a poly(arylene ether) having an intrinsic viscosity of 0.25 to 0.6 dl/g as measured in chloroform at 25° C.;
wherein the nonelastomeric polymer of an alkenylaromatic compound is at least one of polystyrene, impact modified polystyrene, or a non-elastomeric block copolymer composition of styrene and butadiene; and
wherein the impact modifier is a block copolymer of an alkenyl aromatic compound and a diene; wherein the alkenyl aromatic compound is less that 70% by weight of the impact modifier.

14. The article of claim 11, wherein the composition comprises:
(a) 40 to 95 weight percent at least one poly(arylene ether),
(b) 1 to 60 weight percent of at least one nonelastomeric polymer of an alkenylaromatic compound, and
(c) 1 to 20 weight percent of at least one impact modifier wherein the weight percents are based on the total weight of the composition.

15. The article of claim 11, wherein the article is a tray useful for sterilization of surgical, orthopedic, dental, and/or veterinary instruments.

16. The article of claim 11, wherein the article is one of a medical instrument component or a medical instrument handle.
